(19) United States
(12) Patent Application Publication
(10) Pub. No.: US 2025/0381315 A1
(43) Pub. Date: Dec. 18, 2025

(54) PHARMACEUTICAL COMPOSITION FOR WOUND TREATMENT COMPRISING WOUND-COATING MATERIAL AND CHEMOKINE-ADSORBING PARTICLES

(71) Applicant: Seoul National University R&DB Foundation, Seoul (KR)

(72) Inventors: Young Bin CHOY, Seoul (KR); Chang Hee MIN, Seoul (KR); Se Na KIM, Sejong (KR)

(21) Appl. No.: 18/877,495

(22) PCT Filed: Jun. 23, 2023
(86) PCT No.: PCT/KR2023/008730

§ 371 Date: Dec. 20, 2024

(30) Foreign Application Priority Data
Jun. 24, 2022 (KR) 10-2022-0077795

(51) Int. Cl. A61L 26/00; C08L 75/04

(52) U.S. Cl. CPC A61L 26/0019; A61L 26/0066; A61L 26/008; A61L 26/0085; C08L 75/04; A61L 2300/102

(57) ABSTRACT

The present application relates to a pharmaceutical composition for wound treatment. Specifically, the present application concerns a composition including a wound-coating material and chemokine-adsorbing particles to absorb inflammatory chemokines that cause excessive immune responses, thereby allowing active wound healing.

### CROSS-REFERENCE TO RELATED APPLICATION(S)

This application claims priority to and the benefit of Korean Patent Application No. 10-2022-0077795, filed on Jun. 24, 2022, with the Korean Intellectual Property Office, the disclosure of which is incorporated herein by reference in its entirety.

The present application relates to a composition for wound treatment, and more particularly, to a composition comprising a wound dressing and chemokine-adsorbing particles, which absorbs inflammatory chemokines causing excessive immune responses, thereby allowing active wound healing.

### BACKGROUND

Skin is the organ that occupies the largest surface area in our body, which is the organ that protects the muscles and organs in the body from various harmful environments such as external microorganisms, ultraviolet rays, and chemicals, and suppresses moisture evaporation from the human body to prevent dehydration and regulate body temperature. If the skin is wounded due to various causes such as burns, trauma, bedsores, and skin diseases, the skin is unable to perform these important functions properly, which causes a disturbance in the maintenance of homeostasis in the human body. Therefore, the restoration of damaged skin tissue is an important issue, and wound treatment is the basis of all trauma and post-surgery recovery treatment, and is an important process that allows patients to return to their original state through the biological process of repairing damaged skin tissue.

The normal wound healing mechanism goes through four stages: the hemostasis stage after tissue damage, the inflammation stage, the proliferation stage, and the maturation stage. At this time, various growth factors and cells appear at each stage, and extracellular matrices for tissue regeneration are synthesized to repair the damaged site. Normal wound healing process is usually completed at the point in time at which the proliferation stage ends, but if there is a delay at any stage, scars may be generated after recovery, or the wound may progress to a chronic wound that does not heal for several months. Most of these delays occur at the inflammatory stage, and the cause of the delay at the inflammatory stage is pointed to be caused by cytokines, especially chemokines, that are excessively secreted at the wound site.

In order to quickly complete wound treatment and minimize various secondary side effects (infection, progression to chronic wound, scar formation, etc.), wound treatment using appropriate coating material or dressing is essential. Current wound treatment methods include methods that apply disinfectants to the wound, and methods that use various shapes and materials of wound dressings to cover the wound site with absorbent materials to absorb exudate generated from the wound, and maintain an appropriate moist environment at the wound site.

However, these wound dressings have limitations of being passive treatment devices that only alleviate symptoms, while being unable to solve the problem that wound treatment is delayed due to excessive inflammatory response, which leads to scarring and chronic wounds. Therefore, the present inventors have completed a wound dressing which selectively adsorbs the excessively secreted inflammatory chemokines to regulate the concentration of inflammatory chemokines in the wound site, and thereby regulates excessive inflammatory responses occurring in the wound site, and ultimately induces so that normal wound healing mechanism can occur, thereby enabling more fundamental treatment in wound treatment.

### DETAILED DESCRIPTION OF THE INVENTION

### Technical Problem

It is an object of the present application to provide chemokine-absorbing particle and its use in wound treatment.

Specifically, an embodiment provides a composition for chemokine adsorption comprising at least one selected from the group consisting of metal organic framework (MOF) and silica (SiO₂, silicon dioxide, silicic acid).

Another embodiment provides a pharmaceutical composition for wound treatment comprising at least one selected from the group consisting of metal organic framework and silica.

The pharmaceutical composition for wound treatment may further include a wound dressing.

An embodiment provides a pharmaceutical composition for wound treatment comprising a wound dressing and chemokine-absorbing particle. The chemokine-absorbing particle may comprise at least one selected from the group consisting of metal organic framework (MOF), and silica (SiO₂, silicon dioxide, silicic acid).

### Technical Solution

An aspect of the present application relates to the use of chemokine adsorbing particle for treatment of wound.

An embodiment of the present application provides a pharmaceutical composition for wound treatment comprising a wound dressing and chemokine-absorbing particle.

An embodiment of the present application provides the use of chemokine-adsorbing particle in treatment of wound.

An embodiment of the present application provides the use of the chemokine-adsorbed particle in the preparation of a composition for wound treatment.

Another embodiment of the present application provides a method for treating a wound, comprising administering chemokine-adsorbed particle to a subject in need of wound treatment. The method may further comprise identifying the subject in need of wound treatment prior to treatment.

The pharmaceutical composition for wound treatment may be a wound dressing.

The wound dressing includes chemokine-adsorbing particle, which can absorb inflammatory chemokine at a wound site to suppress excessive inflammatory reactions occurring at the wound site.

The chemokine-absorbing particle may be at least one selected from the group consisting of metal organic framework (MOF) and silica (SiO₂, silicon dioxide, silicic acid).

Below, the present application will be described in more detail.

A wound is a type of injury which is a state in which continuity of the normal structure of body tissue is destroyed. The wound can be classified into: bruises, a state in which the capillaries in the subcutaneous tissue are damaged due to external impact to cause an internal bleeding, abrasions, which are wounds in which the skin is peeled off or scraped away due to friction, etc., lacerations, which are wounds in which the skin is irregularly torn by friction or pressure from a machine or blunt object, punctures, which are wounds in which the tissue is penetrated by sharp objects such as nails, needles, wires, or knives, which have small entrances through the tissue but deep internal damage, incisions, which are cut by sharp objects such as knives or pieces of glass, or incised during surgery, and avulsions, which are wounds in which the skin is torn and loosened by a blunt external force.

The wound dressing may refer to a medical device used for the purpose of covering a wound site to prevent contamination, protect the skin, absorb exudate secreted from a wound, and prevent bleeding or loss of body fluids. The wound dressing includes a hydrocolloid type, a foam type, or a film type as an adhesive sheet type, and a hydrogel type as an ointment (gel) type applied to a wound.

The wound dressing may include at least one material selected from the group consisting of polyethylene, polycaprolactone, polyacrylonitrile, polyurethane, polyoxyethylene glycol, polyether, polyethylene oxide, polyvinyl alcohol, polyvinyl pyrrolidone, polyethylene glycol, chitosan, alginate, gelatin, collagen, pectin, and carboxymethyl cellulose.

The wound dressing may take the form of, for example, a hydrocolloid type, a foam type, a film type, or a hydrogel type.

The wound dressing may include an adhesive agent for attaching to the wound site, and the adhesive agent may be selected from the group consisting of polybutylene, polyisoprene, polyisobutylene, isobutylene rubber, polyethylene-propylene rubber, polyethylene-propylenediene-modified rubber, styrene-butadiene-styrene, styrene-isoprene-styrene, styrene-ethylene-propylene-styrene copolymer polymer, styrene-ethylene-butylene-styrene copolymer polymer, polyester, polyamide, epoxy, silicone resin, fluororesin, polystyrene resin, acrylic resin, methacrylic resin, olefin resin or resin derived therefrom, avocado oil, lanolin oil, jojoba oil, mineral oil, silicone oil, tea tree oil, mink oil, isopropyl myristate, petroleum resin, rosin, alkylphenol-acetylene, alkylphenol-aldehyde resin, terpene resin, other hydrocarbon resins such as asphalt and bitumen, polyalkylated novolac resin, xylene-formaldehyde resin, urethane-based adhesive, silicone-based adhesive and acrylic-based adhesive. In a specific embodiment, the adhesive may be mixed into the wound dressing or applied to one or more surfaces of the wound dressing, but is not limited thereto.

Chemokine (chemotactic cytokine) is a type of cytokine as a signal protein secreted by cells, and is a cytokine having chemotactic activity that attracts specific types of cells around the cell that secretes it. Some chemokines play a role in regulating the movement of immune cells in the body to the wound site or the site of pathogen invasion when a wound occurs or a pathogen invades a tissue, thereby mediating an inflammatory response. Chemokine (inflammatory chemokine) that plays a role in regulating inflammatory responses may be at least one selected from the group consisting of TCA-3(T cell activation gene 3), MCP-1(Monocyte chemoattractant protein-1), MIP-1a (Macrophage inflammatory protein alpha), MIP-1B (Macrophage inflammatory protein beta), RANTES (Regulated on activation, normal T cell expressed and secreted), MCP-3 (Monocyte chemoattractant protein-3), GRO-α (Growth regulated oncogene alpha), GRO-β (Growth regulated oncogene beta), ENA-78(Epithelial neutrophil activating protein 78), NAP-2(Neutrophil activating peptide 2), IL-8(Interleukin-8), and SDF-1(Stromal cell derived factor 1).

The composition according to an embodiment, or the wound dressing according to an embodiment may include chemokine-absorbing particles, wherein the chemokine-absorbing particles can adsorb the inflammatory chemokine at the wound site and regulate an inflammatory response occurring at the wound site. As an embodiment, the chemokine-absorbing particles can adsorb inflammatory chemokines that cause an inflammatory response present in the exudate secreted from the wound absorbed by the wound dressing, thereby suppressing an excessive inflammatory response occurring at the wound site. Since the inflammatory chemokine adsorbed to the chemokine-absorbing particles is adsorbed until the wound dressing is replaced, the concentration of the inflammatory chemokine at the wound site is reduced, thereby suppressing an excessive inflammatory response occurring and inducing normal wound treatment.

As used in the present application, the “treatment” means all actions that improve or beneficially change the symptoms of a wounded subject by administering the composition according to an embodiment. This may include the meaning of “prevention” that inhibits or delays the wound, or “amelioration” that at least reduces a parameter associated with a state in which a wound is treated by the composition according to an embodiment, for example, an inflammatory chemokine. The wound may mean a wound or a chronic wound.

The wound may be characterized by secreting a specific chemokine, wherein the chemokine may be at least one selected from the group consisting of TCA-3, MCP-1, MIP-1a, MIP-1B, RANTES, MCP-3, GRO-α, GRO-β, ENA-78, NAP-2, IL-8, and SDF-1.

In an embodiment, the chemokine-adsorbing particle may be at least one selected from the group consisting of metal organic frameworks (MOFs) and silica (SiO₂, silicon dioxide, silicic acid).

The metal-organic framework may refer to a very regular crystalline material in which metal ions or metal clusters are bound to organic ligands acting as linkers through coordinate bonds to form a network. The metal-organic framework can have a wide variety of structures that can be created depending on the type of metal, the morphology of the metal cluster, and the shape or length of the organic ligand, and has a porous crystal structure and a large surface area.

In an embodiment, the metal-organic framework may include zinc (Zn), zirconium (Zr), cobalt (Co), iron (Fe), aluminum (Al), magnesium (Mg), copper (Cu), or nickel (Ni).

In an embodiment, the metal-organic framework may include zirconium. The metal-organic framework including zirconium may be at least one selected from the group consisting of UiO-66(Zr)([Zr₆O₄(OH)₄(C₈H₄O₄)₆], UiO-67(Zr) [Zr₆O₄(OH)₄(O₂C(C₆H₄)₂CO₂)₆]), NU-1000(Zr)[Zr₆(μ₃—O)₄(μ₃—OH)₄(H₂O)₄(OH)₄(TBAPy)₂]; TBAPy=4,4,4,4-(pyrene-1,3,6,8-tetrayl)tetrabenzoic acid]), MOF-808(Zr)([Zr₆O₄(OH)₄(BTC)₂(HCOO)₆]; BTC=1,3,5-benzenetricarboxylic acid), PCN-223(Zr)([Zr₆O₄(OH)₄(TCPP) 3]), and PCN-222(Zr)([Zr₆(μ₃—O)₈(OH)₈(TCPP)₂]; TCPP=Tetrakis (4-carboxyphenyl)porphyrin).

In an embodiment, the PCN-222(Zr)([Zr₆(μ₃—O)₈(OH)₈(TCPP)₂]; TCPP=Tetrakis(4-carboxyphenyl)porphyrin) is a metal organic framework having zirconium (Zr) as a metal ion and TCPP (tetrakis(4-carboxyphenyl)porphyrin, CAS No. 14609-54-2) as a ligand.

The silica is a structure in which silicon oxide molecules form an ultrafine meshwork and many holes and spaces exist between the particles, and has a large surface area and can adsorb small molecules well.

The chemokine adsorption particles (metal organic frameworks or silica) has a porous structure, so that the chemokine can be attached to the pore space by a method such as physical adsorption or chemical adsorption.

The chemokine-adsorbing particles may selectively bind to the chemokine. In an embodiment, the chemokine-adsorbing particles may bind to the chemokine by electrostatic attraction. In an embodiment, the chemokine-adsorbing particles are negatively charged and the chemokine is positively charged, and can bind to each other by electrostatic attraction.

In an embodiment, the positively charged chemokine may be at least one selected from the group consisting of TCA-3, MCP-1, RANTES, MCP-3, GRO-α, ENA-78, NAP-2, IL-8 and SDF-1.

The composition according to an embodiment absorbs inflammatory chemokines better than existing wound dressings that simply provide a moist environment, and therefore, not only has a better wound treatment effect, but may also not cause side effects even if attached to the wound site for a long time.

In an embodiment, in order to effectively bind the wound dressing and the chemokine-adsorbing particles, a method of binding a linker molecule having an active group (—OH, —COOH, —NH₂) to the surface of the wound dressing, a method of directly synthesizing particles on the surface of the wound dressing using a monomer of the adsorbing particles, a method of fabricating the wound dressing by adding the adsorbing particles together, and a method of treating the wound dressing with oxygen plasma, etc. may be used, but is not limited thereto. In an embodiment, the amount of the chemokine-adsorbing particles (metal organic frameworks or silica) included in the composition may be in a range such that the amount of chemokine-adsorbing particles applied per unit area (cm²) of the wound site is 0.0001 to 1000 mg.

In an embodiment, the amount of the chemokine-adsorbing particles (metal organic frameworks or silica) included in the composition in an amount of 0.0001 to 1 mg, 0.0001 to 0.1 mg, 0.0001 to 0.01 mg, 0.0001 to 0.001 mg, 0.001 to 1 mg, 0.001 to 0.1 mg, 0.001 to 0.01 mg, 0.0025 to 1 mg, 0.0025 to 0.1 mg, 0.0025 to 0.01 mg, 0.005 to 1 mg, 0.005 to 0.75 mg, 0.005 to 0.625 mg, 0.005 to 0.5 mg, 0.005 to 0.25 mg, 0.005 to 0.1 mg, 0.005 to 0.05 mg, 0.005 to 0.025 mg, 0.005 to 0.01 mg, 0.005 to 0.0075 mg, 0.0075 to 1 mg, 0.0075 to 0.75 mg, 0.0075 to 0.625 mg, 0.0075 to 0.5 mg, 0.0075 to 0.25 mg, 0.0075 to 0.1 mg, 0.0075 to 0.05 mg, 0.0075 to 0.025 mg, 0.0075 to 0.01 mg, 0.01 to 1 mg, 0.01 to 0.75 mg, 0.01 to 0.625 mg, 0.01 to 0.5 mg, 0.01 to 0.25 mg, 0.01 to 0.1 mg, 0.01 to 0.05 mg, 0.01 to 0.025 mg, 0.025 to 1 mg, 0.025 to 0.75 mg, 0.025 to 0.625 mg, 0.025 to 0.5 mg, 0.025 to 0.25 mg, 0.025 to 0.1 mg, 0.025 to 0.05 mg, 0.05 to 1 mg, 0.05 to 0.75 mg, 0.05 to 0.625 mg, 0.05 to 0.5 mg, 0.05 to 0.25 mg, 0.05 to 0.1 mg, 0.1 to 1 mg, 0.1 to 0.75 mg, 0.1 to 0.625 mg, 0.1 to 0.5 mg, 0.1 to 0.25 mg, 0.25 to 1 mg, 0.25 to 0.75 mg, 0.25 to 0.625 mg, 0.25 to 0.5 mg, 0.5 to 1 mg, 0.5 to 0.75 mg, 0.5 to 0.625 mg, 0.625 to 1 mg, 0.625 to 0.75 mg, or 0.75 to 1 mg, per unit area (cm²) of the wound site. In an embodiment, the metal organic frameworks may be PCN-222(Zr).

In an embodiment, taking into consideration the problem of cytotoxicity and wound treatment effects, it is preferable to adjust the amount of the metal-organic frameworks in the composition to the above range.

In an embodiment, the silica (SiO₂) may be included in the composition in an amount of 0.01 to 1000 mg, 0.01 to 500 mg, 0.01 to 100 mg, 0.01 to 10 mg, 0.01 to 1 mg, 0.01 to 0.1 mg, 0.1 to 1000 mg, 0.1 to 500 mg, 0.1 to 100 mg, 0.1 to 10 mg, 0.1 to 1 mg, 0.25 to 500 mg, 0.25 to 200 mg, 0.25 to 175 mg, 0.25 to 150 mg, 0.25 to 125 mg, 0.25 to 100 mg, 0.25 to 50 mg, 0.25 to 20 mg, 0.25 to 10 mg, 0.25 to 5 mg, 0.25 to 1 mg, 0.5 to 500 mg, 0.5 to 200 mg, 0.5 to 175 mg, 0.5 to 150 mg, 0.5 to 125 mg, 0.5 to 100 mg, 0.5 to 50 mg, 0.5 to 20 mg, 0.5 to 10 mg, 0.5 to 5 mg, 0.5 to 1 mg, 0.75 to 500 mg, 0.75 to 200 mg, 0.75 to 175 mg, 0.75 to 150 mg, 0.75 to 125 mg, 0.75 to 100 mg, 0.75 to 50 mg, 0.75 to 20 mg, 0.75 to 10 mg, 0.75 to 5 mg, 0.75 to 1 mg, 1 to 500 mg, 1 to 200 mg, 1 to 175 mg, 1 to 150 mg, 1 to 125 mg, 1 to 100 mg, 1 to 50 mg, 1 to 20 mg, 1 to 10 mg, 1 to 5 mg, 5 to 500 mg, 5 to 200 mg, 5 to 175 mg, 5 to 150 mg, 5 to 125 mg, 5 to 100 mg, 5 to 50 mg, 5 to 20 mg, 5 to 10 mg, 10 to 500 mg, 10 to 200 mg, 10 to 175 mg, 10 to 150 mg, 10 to 125 mg, 10 to 100 mg, 10 to 50 mg, 10 to 20 mg, 20 to 500 mg, 20 to 200 mg, 20 to 175 mg, 20 to 150 mg, 20 to 125 mg, 20 to 100 mg, 20 to 50 mg, 50 to 500 mg, 50 to 200 mg, 50 to 175 mg, 50 to 150 mg, 50 to 125 mg, 50 to 100 mg, 100 to 500 mg, 100 to 200 mg, 100 to 175 mg, 100 to 150 mg, 100 to 125 mg, 125 to 500 mg, 125 to 200 mg, 125 to 175 mg, 125 to 150 mg, 150 to 500 mg, 150 to 200 mg, 150 to 175 mg, 175 to 500 mg, 175 to 200 mg or 200 to 500 mg, per area (cm²) of the wound site.

In an embodiment, taking into consideration the problem of cytotoxicity and wound treatment effects, it is preferable to adjust the amount of silica in the composition to the above range.

The composition according to an embodiment can be attached to a wound site to adsorb inflammatory chemokines within a short period of time, thereby inducing normal wound treatment. In an embodiment, the composition according to the present application can be treated to a wound site for 48 hours or less, 24 hours or less, 12 hours or less, 6 hours or less, 4 hours or less, 3 hours or less, 2 hours or less, 1 hour to 2 hours, 1 hour to 3 hours, 1 hour to 4 hours, 1 hour to 6 hours, 1 hour to 12 hours, 1 hour to 24 hours, 1 hour to 48 hours, 2 hours to 3 hours, 2 hours to 4 hours, 2 hours to 6 hours, 2 hours to 12 hours, 2 hours to 24 hours, 2 hours to 48 hours, 3 hours to 4 hours, 3 hours to 6 hours, 3 hours to 12 hours, 3 hours to 24 hours, 3 hours to 48 hours, 4 hours to 6 hours, 4 hours to 12 hours, 4 hours to 24 hours, 4 hours to 48 hours, 6 hours to 12 hours, 6 hours to 24 hours, 6 hours to 48 hours, 12 hours to 24 hours, 12 hours to 48 hours or 24 hours to 48 hours.

Another embodiment provides a composition for chemokine adsorption comprising at least one selected from the group consisting of metal organic frameworks (MOFs) and silica (SiO₂, silicon dioxide, silicic acid).

The composition for chemokine adsorption may further include a wound dressing.

The metal organic frameworks may be at least one selected from the group consisting of UiO-66(Zr), UiO-67(Zr), NU-1000(Zr), MOF-808(Zr), PCN-223(Zr), and PCN-222(Zr).

The chemokine may be at least one selected from the group consisting of TCA-3, MCP-1, MIP-1a, MIP-1B, RANTES, MCP-3, GRO-α, GRO-β, ENA-78, NAP-2, IL-8, and SDF-1.

The details of the metal organic frameworks, silica, and chemokine are as described above.

Yet another embodiment provides a pharmaceutical composition for wound treatment comprising at least one selected from the group consisting of metal-organic frameworks and silica.

The metal-organic frameworks may be at least one selected from the group consisting of UiO-66(Zr), UiO-67(Zr), NU-1000(Zr), MOF-808(Zr), PCN-223(Zr), and PCN-222(Zr).

The wound may be characterized by secreting a specific chemokine, and the chemokine may be at least one selected from the group consisting of TCA-3, MCP-1, MIP-1a, MIP-1B, RANTES, MCP-3, GRO-α, GRO-β, ENA-78, NAP-2, IL-8, and SDF-1.

The details of the metal-organic frameworks, silica, and chemokine are as described above. The amount of the metal-organic frameworks or silica contained in the pharmaceutical composition for wound treatment is as described above.

The method of application of the pharmaceutical composition may be any method that has been used heretofore, for example, it may be applied to the skin where a wound has occurred. The composition may be used by being formulated into a transdermal agent, such as a suspension, emulsion, aerosol, ointment, patch, gel, covering material, etc., according to a conventional method, and used, but is not limited thereto. In an embodiment, the composition may further include a transdermal agent, such as a patch, gel, or wound dressing.

### Advantageous Effects

The wound dressing and the pharmaceutical composition for wound treatment comprising chemokine-absorbing particles according to the present application absorb inflammatory chemokines that cause excessive immune responses, thereby allowing active wound healing.

### BRIEF DESCRIPTION OF THE DRAWING

FIG. 1 is a schematic diagram showing a wound dressing containing chemokine-adsorbed particles.

FIG. 2 is a schematic diagram showing that a wound dressing containing chemokine-adsorbed particles absorbs inflammatory chemokines at the wound site and heals the wound.

FIG. 3 is a graph showing cell viability after an indirect cell cytotoxicity test using MOF or silica (SiO₂) particles.

FIG. 4 is a graph showing the adsorption rate of chemokines MCP-1 and IL-8 over time when MOF is added in various amounts.

FIG. 5 is a graph showing the adsorption rate of chemokines MCP-1 and IL-8 over time when SiO₂ particles are added in various amounts.

FIG. 6 is a graph showing the adsorption rate of chemokines MCP-1 and IL-8 over time during MOF dressing.

FIG. 7 is a representative image of wound healing in a group in which a MOF dressing was applied to a mouse wound model and in a control group.

FIG. 8 is a graph showing the percentage of wound cloth in a group in which a MOF dressing was applied to a mouse wound model and in a control group.

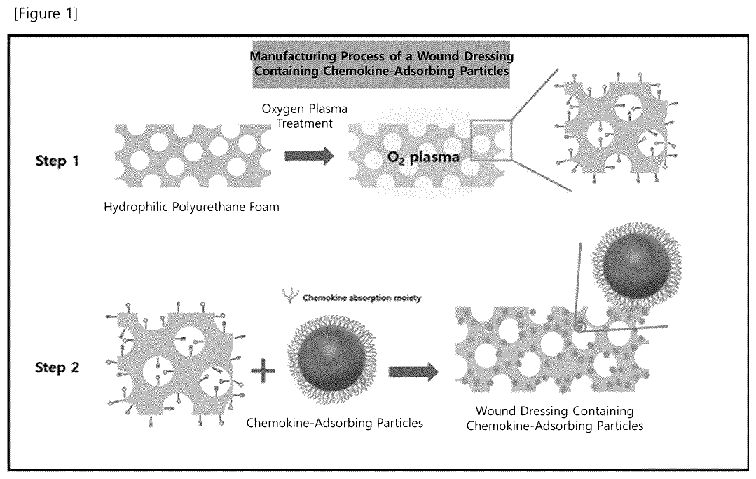

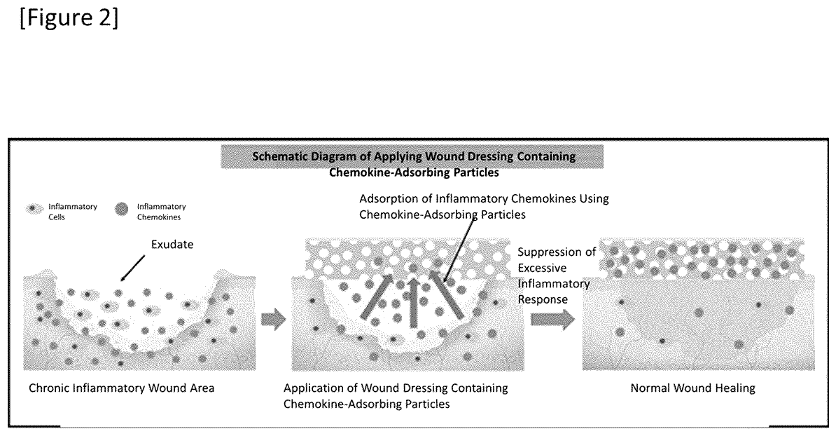

### DETAILED DESCRIPTION OF THE EMBODIMENTS

Hereinbelow, the present application will be described in detail. Meanwhile, each of the explanations and exemplary embodiments disclosed herein can be applied to other explanations and exemplary embodiments. That is, all combinations of various factors disclosed herein belong to the scope of the present application. Furthermore, the scope of the present application should not be limited by the specific disclosure provided hereinbelow. Additionally, those of ordinary skill in the art may be able to recognize or confirm, using only conventional experimentation, many equivalents to the particular aspects of the present application described herein. Furthermore, it is also intended that these equivalents be included in the present application.

### 1. Cytotoxicity Test of Chemokine-Adsorbing Particles

### 1-1. Experimental Preparation

In this example, PCN-222(Zr) and silica (SiO₂) were used as organic metal frameworks which are chemokine-adsorbing particles. The PCN-222(Zr)([Zr₆(μ₃—O)₈(OH)₈(TCPP)₂]; TCPP=Tetrakis(4-carboxyphenyl)porphyrin) used was synthesized using zirconyl chloride octahydrate and TCPP obtained from Sigma Aldrich and TCI. The silica used was obtained from ES Food Ingredients Co., Ltd.

In this example, human MCP-1(monocyte chemoattractant protein-1) and IL-8(interleukin-8) were used as chemokines, and each was obtained from Prospec and used.

### 1-2. Cytotoxicity Test of Chemokine-Adsorbing Particles

Cytotoxicity experiments were performed to examine the effects of chemokine-adsorbing particles on cell viability. Cytotoxicity tests were performed using the in vitro cytotoxicity test on extracts method. Particles and RPMI medium were placed in a 15 mL conical tube, and the eluate, eluted at 37° C. for 48 hours, was used as the test solution (PCN-222; 2 mg/ml, silica; 200 mg/ml). Cytotoxicity tests were performed using the L929 mouse fibroblast cell line. L929 cells were cultured in RPMI 1640 medium containing 10% fetal bovine serum, 1% penicillin-streptomycin at 37° C. under a humidified atmosphere containing 5% CO₂. To test cytotoxicity, 100 μl of cell suspension (5.0×10⁴ cells/ml) was seeded into each well of a 96-well cell culture plate. After 24 hours, the eluate was added to each well at various concentrations (PCN-222; 0.001, 0.005, 0.01, 0.05, 0.1, 0.25, 0.75, and 1 mg/ml, silica; 0.01, 0.05, 0.1, 0.5, 1, 5, 10, 20, 50, 100, 150, and 200 mg/ml). Then, the 96-well cell culture plate was cultured at 37° C. for 24 hours under a 5% CO₂ humidified atmosphere. To assess cell viability, 10 μl of Ez-Cytox solution was added to each well and the plate was further incubated at 37° C. for 1 hour. The plate was read for absorbance at 450 and 600 nm using a microplate reader. The cell viability was calculated using the following Equation.

$$Cell viability ( \% ) = ( Absorbance at 450 nm of wells with eluate - Absorbance at 600 nm of wells with eluate ) / ( Absorbance at 450 nm of wells without eluate - Absorbance at 600 nm of wells without eluate ) \times 100 \qquad ( Equation 1 )$$

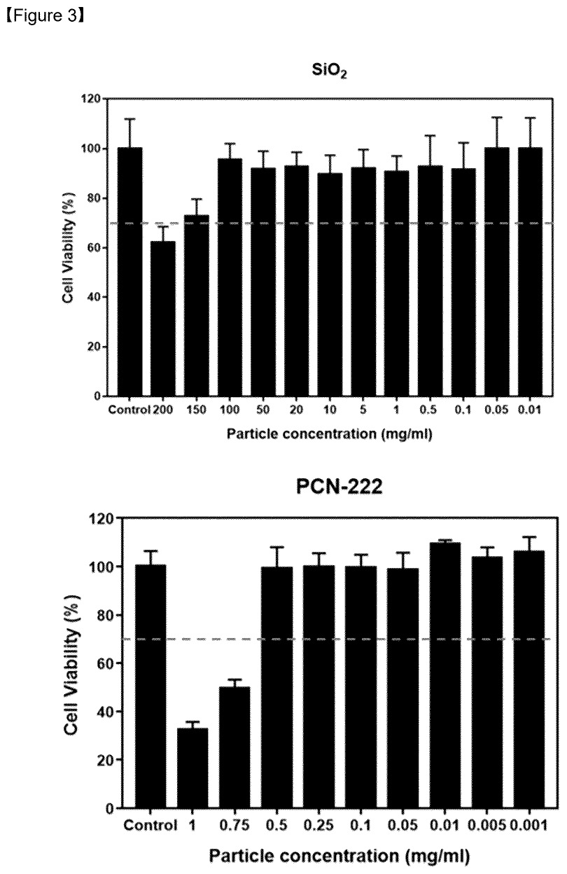

The results of the cytotoxicity experiment are shown in FIG. 3.

As shown in FIG. 3, when PCN-222 was used at a concentration of 0.75 mg/ml, the cell viability was less than 50%, and when it was used at a concentration of 1 mg/ml, the cell viability was less than 40%. In addition, when silica was used at a concentration of 200 mg/ml, the cell viability was less than 70%.

When PCN-222 was used in a concentration range of 0.5 mg/ml or less and silica in a concentration range of 150 mg/ml or less, it exhibited the cell viability of 70% or more, confirming a stable concentration range for cell survival.

2. Chemokine adsorption experiment of PCN-222(Zr)

Chemokine adsorption experiments of PCN-222(Zr) were performed using RPMI 1640 medium containing 2% FBS (fetal bovine serum). Chemokine (human MCP-1 or IL-8) solution (100 μg/ml) was diluted to concentrations of 2 ng/ml and 2000 ng/ml in the culture medium, respectively. PCN-222(Zr) prepared in Example 1-1 was made into a suspension at a concentration of 1 mg/ml using RPMI 1640 medium, and then diluted by concentrations (0.002, 0.005, 0.01, 0.02, 0.05, 0.1, 0.2, 0.5, 1 mg/ml), placed by 0.5 ml each in a 1.5 ml conical tube, and then 0.5 ml of a chemokine (human MCP-1 or IL-8) solution was added and mixed well with a vortex mixer. Then, the mixture was incubated at room temperature for several hours (1, 6, 24, and 48 hours). The mixture was centrifuged at 13,000 rpm for 5 minutes to separate the supernatant. The separated supernatant was analyzed by ELISA, and the adsorption rate (%) was calculated according to the following Equation 2.

$$Absorbance rate ( \% ) = ( Amount of chemokine initially added - Amount of chemokine remaining in the supernatant ) / ( Amount of chemokine initially added ) \times 100 \qquad ( Equation 2 )$$

ELISA was performed using ThermoFisher Scientific's Human LI-8 uncoated ELISA and Human CCL2(MCP-1) Uncoated ELISA. A solution of the capture antibody diluted in a coating buffer was placed in a Corning Costar 9018 ELISA plate at 100 μl per well, and then incubated at 4° C. for 24 hours, followed by coating. Then, each well was washed 3 times using a washing solution, and 200 μl of ELISA diluent was added to each well, which was then incubated at room temperature for 1 hour to block the wells. After 1 hour, washing was performed with a wash solution, and 100 μl of the standard solution and sample solution were added to the designated wells, and incubated at room temperature for 2 hours. After incubation, the mixture was washed 3 to 5 times using the washing solution, and 100 μl of detection antibody diluted in detection buffer was added to each well, and incubated for 1 hour. After 1 hour, washing was performed 3 to 5 times using the washing solution, and 100 μl of streptavidin-HRP (Horseradish peroxide) solution was added to each well, and then incubated at room temperature for 30 minutes. Then, the wells were washed 5 to 7 times using the washing solution, and 100 μl of TMB (Tetramethylbenzidine) solution was added to each well, and then incubated for 15 minutes. Then, 100 μl of a stop solution (1 M H₃PO₄ solution) was added to each well, and the absorbance at 450 nm and 570 nm was measured using a microplate reader to determine the amount of chemokine remaining in the supernatant.

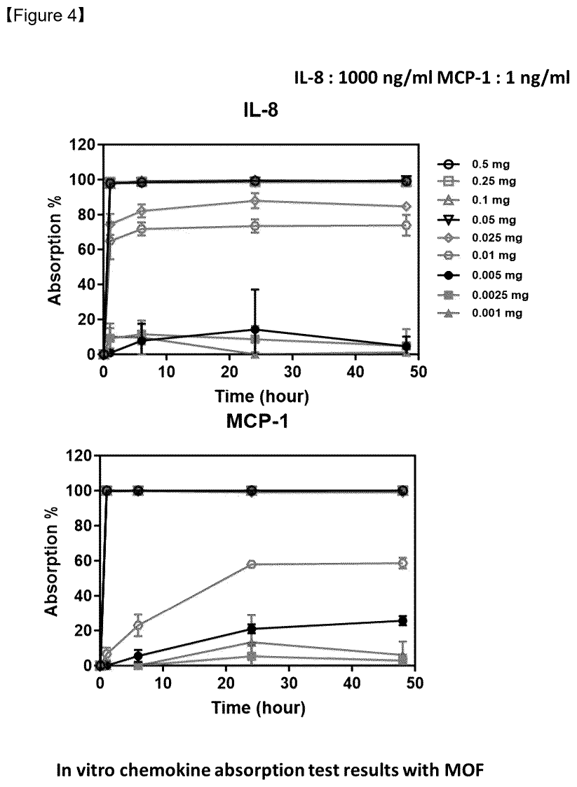

The results of the chemokine adsorption experiment of PCN-222(Zr) are shown in FIG. 4. As shown in FIG. 4 (lower diagram), as a result of the MCP-1 adsorption experiment, when the PCN-222 concentration was 0.025 mg/ml or more, PCN-222 showed an adsorption rate of nearly 100% in 1 hour, and the adsorbed chemokine was not released but remained in a state adsorbed continuously for up to 48 hours. When the concentration of PCN-222 was 0.01 mg/ml or more, PCN-222 showed an adsorption rate of 50% or more for 24 hours. Furthermore, as a result of the IL-8 adsorption experiment (upper diagram of FIG. 4), when the concentration of PCN-222 was 0.05 mg/ml or more, PCN-222 showed an adsorption rate of nearly 100% in 1 hour, and the adsorbed chemokine was not released and was continuously in a state adsorbed for up to 48 hours. When the concentration of PCN-222 was 0.01 mg/ml or more, PCN-222 showed an adsorption rate of 50% or more for 24 hours.

### 3. Chemokine Adsorption Experiment of Silica

RPMI 1640 medium was prepared in the same manner as in Example 2, and chemokines (human MCP-1 or IL-8) were diluted. The silica prepared in Example 1-1 was made into a suspension at a concentration of 20 mg/ml using RPMI 1640 medium, then diluted by concentrations (0.2, 2, 10, 20 mg/ml) and placed 0.5 ml each in a 1.5 ml conical tube. Next, 0.5 ml of chemokine solution was added and mixed well with a vortex mixer. Then, the mixture was incubated at room temperature for several hours (1, 6, 24, and 48 hours). The mixture was centrifuged at 13,000 rpm for 5 minutes to separate the supernatant. The separated supernatant was analyzed by ELISA, and the adsorption rate (%) was calculated according to Equation 2.

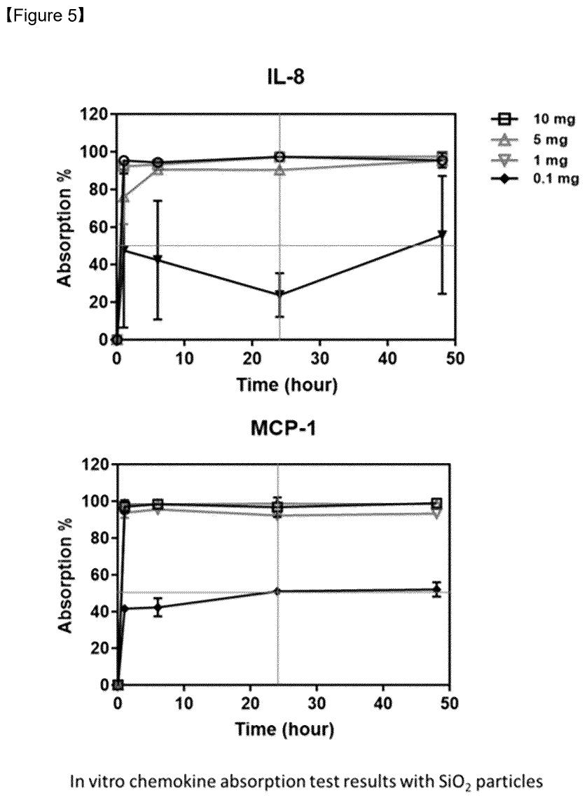

The results of the chemokine adsorption experiment of silica are shown in FIG. 5. As shown in FIG. 5, as a result of the MCP-1 adsorption experiment (lower diagram of FIG. 5), when the silica concentration was 1 mg/ml or more, silica showed an adsorption rate of nearly 100% for 24 hours, and most of the adsorbed chemokines were not released for up to 48 hours and remained in a state adsorbed on the chemokine adsorbed particles. In addition, as a result of the IL-8 adsorption experiment (upper diagram of FIG. 5), when the silica concentration was 5 mg/ml or more, silica showed an adsorption rate of nearly 100% in 1 hour, and when the silica concentration was 1 mg/ml or more, SiO₂ particles showed an adsorption rate of nearly 100% for 24 hours, and most of the adsorbed chemokines were not released for up to 48 hours and remained in a state adsorbed on the chemokine adsorbed particles.

### 4. Chemokine Adsorption Experiment of MOF Dressing

### 4-1. Manufacturing of MOF Dressing Foam

In order to bind the produced PCN-222(Zr) to polyurethane foam, polyurethane foam (PU foam) was treated with oxygen plasma. PU foam treated with oxygen plasma has many oxygen radicals (—OH, —O) on its surface, and such radicals were highly activated to form hydrogen bonds with other functional groups. Using these radicals, other MOFs including PCN-222(Zr) could be bound to PU foam to produce MOF-bound PU foam (MOF-PU foam). The polyurethane foam used for the production of MOF dressing was hydrophilic polyurethane foam so that it could absorb the exudate from the wound well, and the chemokines in the exudate could be well adsorbed by PCN-222(Zr) in the polyurethane foam.

To prepare PU foam combined with oxygen plasma treatment and PCN-222(Zr), an expandable plasma cleaner available from Harrick Plasma was used. Polyurethane foam with a diameter of 10 mm was placed in the device, vacuumed, and treated with oxygen plasma for 1 minute. The polyurethane foam treated with plasma was immediately immersed in 30 ml of PCN-222(Zr) suspension with a concentration of 0.5-0.05 mg/ml, and reacted for stirring for 5 minutes. The reacted polyurethane foam was washed repeatedly for 5 minutes using distilled water for a total of 5 times, and then dried in an oven at 40° C. for one day to produce PU foam combined with PCN-222(Zr). The content of MOF in the MOF dressing was measured using ICP-OES. MOF dressing was placed in 10 ml of a 4:1 solution of nitric acid: hydrofluoric acid (HNO₃: HF), heated for 6 hours to ionize the metal clusters in the MOF, and then the solution was measured through ICP-OES to quantify the amount of metal ions, which were converted to quantify the amount of MOF contained in the MOF dressing.

### 4-2. Chemokine Adsorption Experiment of MOF Dressing

A chemokine adsorption experiment of MOF dressing in which PCN-222(Zr) was bound to polyurethane foam (PU foam) was performed.

RPMI 1640 medium was prepared in the same manner as in Example 2, and chemokines (human MCP-1 or IL-8) were diluted to 2 ng/ml and 2000 ng/ml, respectively. PU foam with a diameter of 10 mm was added to a 15 ml conical tube. 0.785 ml of MCP-1 solution and 0.785 ml of IL-8 solution were added to the conical tube.

The conical tube was incubated at room temperature for several hours (1, 6, 24, and 48 hours). The supernatant was separated using a micropipette, and centrifuged at 13,000 rpm for 5 minutes to obtain a purified supernatant. The concentration of the supernatant was analyzed by ELISA, and the adsorption rate (%) was calculated according to Equation 1.

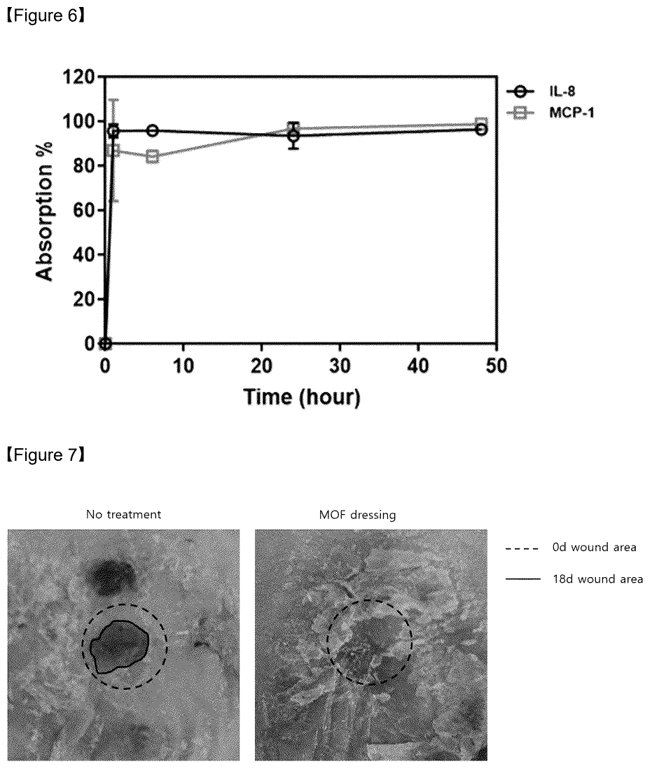

The results of the chemokine adsorption experiment of the MOF dressing are shown in FIG. 6. As shown in FIG. 6, the chemokine adsorption results showed that the MOF dressing adsorbed more than 90% of MCP-1 and IL-8 in 1 hour.

### 5. In Vivo Wound Healing Evaluation Experiment

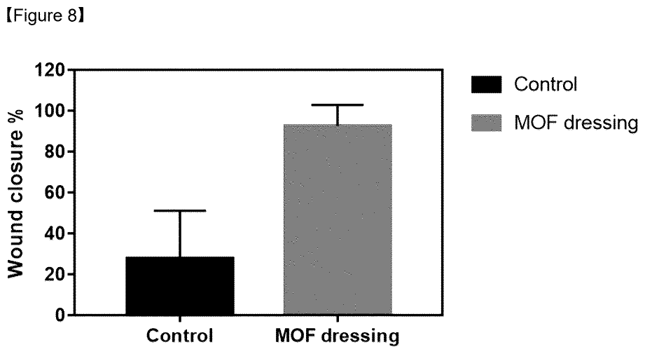

The representative images and wound closure % of each wound healing evaluation experimental group are shown in FIGS. 7 and 8, respectively.

MOF-dressing was prepared in the same manner as in Example 4-1, and sterilized using EO gas sterilization method to prepare MOF-dressing to be used in the in vivo wound healing evaluation experiment. For wound induction, C57BLKS/J-db/db mice purchased from Japan SLC were anesthetized using an inhalation anesthesia device, and then the dorsal skin was incised using a 6 mm diameter circular biopsy punch. The experimental animals were randomly selected and divided into two groups, each of which was a control group that was not treated after wound induction and a group that was treated with MOF-dressing.

To evaluate wound healing, the wound sites were photographed in all animals on the 18th day after wound induction, and the wound sites were quantitatively evaluated using the Image J program. Wound closure % was calculated using the following Equation 3.

$$Wound closure \% = 100 - [ ( Wound size on the 18 th day ) / ( Wound size on the 0 th day ) ⋆ 100 ] \qquad ( Equation 3 )$$

As a result of comparing the wound sites on the 18th day of the experiment, the group treated with MOF dressing showed a significantly narrower wound area than the control group. As a result of comparing the wound closure % between the two groups, the MOF-dressing group showed a wound closure of 92.6±10.1% and the control group showed a wound closure of 27.7±23.4%, respectively. These results showed that the application of MOF dressing exhibits a significant effect on wound healing.

1. A pharmaceutical composition for wound treatment comprising a wound dressing and chemokine-absorbing particle.

2. The pharmaceutical composition for wound treatment according to claim 1, wherein the wound dressing is a hydrocolloid type, a foam type, a film type, or a hydrogel type, comprising at least one material selected from the group consisting of polyethylene, polycaprolactone, polyacrylonitrile, polyurethane, polyoxyethylene glycol, polyether, polyethylene oxide, polyvinyl alcohol, polyvinyl pyrrolidone, polyethylene glycol, chitosan, alginate, gelatin, collagen, pectin, and carboxymethyl cellulose.

3. The pharmaceutical composition for wound treatment according to claim 1, wherein the wound secretes chemokine.

4. The pharmaceutical composition for wound treatment according to claim 3, wherein the chemokine is at least one selected from the group consisting of TCA-3, MCP-1, MIP-1a, MIP-1B, RANTES, MCP-3, GRO-α, GRO-β, ENA-78, NAP-2, IL-8, and SDF-1.

5. The pharmaceutical composition for wound treatment according to claim 1, wherein the chemokine-absorbing particle is at least one selected from the group consisting of metal organic framework, and silica.

6. The pharmaceutical composition for wound treatment according to claim 5, wherein the metal organic framework is at least one selected from the group consisting of UiO-66(Zr), UiO-67(Zr), NU-1000(Zr), MOF-808(Zr), PCN-223(Zr), and PCN-222(Zr).

7. The pharmaceutical composition for wound treatment according to claim 5, wherein:
the pharmaceutical composition adsorbs at least one chemokine selected from the group consisting of TCA-3, MCP-1, RANTES, MCP-3, GRO-α, ENA-78, NAP-2, IL-8, and SDF-1.

8. A method for treating a wound, comprising administering chemokine-adsorbed particle to a subject in need of wound treatment.

9. The method for treating a wound according to claim 8, wherein the wound secretes chemokine.

10. The method for treating a wound according to claim 9, wherein the chemokine is at least one selected from the group consisting of TCA-3, MCP-1, MIP-1a, MIP-1B, RANTES, MCP-3, GRO-α, GRO-β, ENA-78, NAP-2, IL-8, and SDF-1.

11. The method for treating a wound according to claim 9, wherein the chemokine is at least one selected from the group consisting of MCP-1 and IL-8.

12. The method for treating a wound according to claim 8, wherein the chemokine-absorbing particle comprises at least one selected from the group consisting of metal organic framework and silica.

13. The method for treating a wound according to claim 12, wherein the metal organic framework is at least one selected from the group consisting of UiO-66(Zr), UiO-67(Zr), NU-1000(Zr), MOF-808(Zr), PCN-223(Zr) and PCN-222(Zr).

14. The method for treating a wound according to claim 13, wherein the metal organic framework is PCN-222(Zr).

15. The pharmaceutical composition for wound treatment according to claim 3, wherein the chemokine is at least one selected from the group consisting of MCP-1 and IL-8.

16. The pharmaceutical composition for wound treatment according to claim 5, wherein the metal organic framework is PCN-222(Zr).

17. The method for treating a wound according to claim 8, wherein the chemokine-adsorbed particle is administered with a wound dressing.

18. The method for treating a wound according to claim 17, wherein the wound dressing is a hydrocolloid type, a foam type, a film type, or a hydrogel type, comprising at least one material selected from the group consisting of polyethylene, polycaprolactone, polyacrylonitrile, polyurethane, polyoxyethylene glycol, polyether, polyethylene oxide, polyvinyl alcohol, polyvinyl pyrrolidone, polyethylene glycol, chitosan, alginate, gelatin, collagen, pectin, and carboxymethyl cellulose.

19. A method for absorbing a chemokine, comprising applying a chemokine-adsorbed particle to a subject in need of absorbing a chemokine.

20. The method for absorbing a chemokine according to claim 19, wherein the chemokine-adsorbed particle is at least one selected from the group consisting of metal organic framework, and silica.
